(19) United States
(12) Patent Application Publication
(10) Pub. No.: US 2015/0374240 A1
(43) Pub. Date: Dec. 31, 2015

(54) Heart Rate Inference Based On Accelerometer And Cardiac Model

(71) Applicant: Salutron, Inc., Fremont, CA (US)

(72) Inventor: Yong Jin Lee, Palo Alto, CA (US)

(21) Appl. No.: 14/315,643

(22) Filed: Jun. 26, 2014

(51) Int. Cl. A61B 5/0205; A61B 5/00; A61B 5/11

(52) U.S. Cl. CPC A61B 5/0205; A61B 5/1118; A61B 5/4866; A61B 5/7221; A61B 5/7278; A61B 5/6801; A61B 5/742; A61B 5/024

(57) ABSTRACT

An activity monitor determines whether the current value of a user's heart rate from a heart rate sensor is correct based on a motion-based heart rate and an associated spread which are determined by a model. The model provides a probability density function (PDF) of the heart rate based on confidence levels of previous heart rate values. The spread of the PDF is inversely proportional to the confidence levels. The confidence level may be based on an amplitude of a spectral peak relative to a noise floor. Multiple spectral peaks within the spread can be processed based on a shape of the PDF to determine which peak to use as the current value from the heart rate sensor. Either the value from the heart rate sensor or the motion-based heart rate is provided as the current heart rate.

### BACKGROUND

Activity monitors or actigraphs have become popular as a tool for promoting exercise and a healthy lifestyle. An activity monitor can include an accelerometer which can measure motions such as steps taken while walking or running, and estimate an amount of calories used. Moreover, user-specific information such as age, gender, height and weight can be used to tailor the estimate to the user. Such monitors can be worn on the wrist, belt or arm, for instance, or carried in the pocket. The monitor can be worn during an intended workout period or as a general, all day, free living monitor, where the user may perform specific exercises at some times while going about their daily activities at other times, e.g., including sitting, standing and sleeping. An activity monitor can also include a heart rate sensor. Heart rate sensors are also used to monitor individuals, typically during an exercise session in which the user tries to maintain a target heart rate. There is need to continue the development of such monitors.

### SUMMARY

Devices and techniques are provided herein which provide a heart rate measurement of a user with increased reliability. In one aspect, a cardiac (heart) model is used to determine a current heart rate value for a user based on factors such as one or more prior heart rate values, an energy expenditure rate based on motion data from a motion sensor, and one or more physiological parameters of the user. The current heart rate value can be an output from the model or a current value from a heart rate sensor worn by the use. In one approach, the current value from the heart rate sensor may be used if it is consistent with the output from the model. Or, the output from the model can be used if the current value from the heart rate sensor appears to be incorrect in comparison to a motion-based heart rate determined by the model.

In an example implementation, the model determines a motion-based heart rate as an estimate of the actual heart rate, and a spread of the estimate, e.g., a +/− range. In one approach, the estimate and spread are provided by a probability density function. The motion-based heart rate can be used as the heart rate when the current value from the heart rate sensor appears to be incorrect. Moreover, the spread can be relatively narrow when confidence levels associated with the one or more prior heart rate values are relatively large. To provide an efficient implementation, the spread can be selected from a plurality of predetermined spreads, such as based on the above-mentioned confidence levels.

As a result, the reliability of the heart rate data from the heart rate sensor can be checked. In cases where the heart rate data is not reliable, such as when the sensor is moving or not in good contact with the user's body, a reliable alternative heart rate value can be provided from the model. The heart rate value can be output such as for display on a display device or storage in memory for subsequent display or processing.

This summary is provided to introduce a selection of concepts in a simplified form that are further described below in the description. This summary is not intended to identify key features or essential features of the claimed subject matter, nor is it intended to be used to limit the scope of the claimed subject matter.

### BRIEF DESCRIPTION OF THE DRAWINGS

In the drawings, like-numbered elements correspond to one another.

FIG. 1A depicts a front view of an example activity monitor.

FIG. 1B depicts a rear view of the activity monitor of FIG. 1A.

FIG. 1C depicts an example block diagram of the activity monitor of FIG. 1A.

FIG. 2 depicts a software block diagram of an implementation of the heart rate model 137 of FIG. 1C.

FIG. 3A depicts a flowchart of a process for determining heart rate using the heart rate model 137 of FIG. 2.

FIG. 3B depicts a flowchart of a process for use in step 307 of FIG. 3A for determining an energy expenditure rate of a user based on motion data from a motion sensor.

FIG. 3C depicts a flowchart of a process for use in step 311 of FIG. 3A for determining a spread based on confidence levels.

FIG. 3D depicts a flowchart of a process for use in step 316 of FIG. 3A for adjusting a heart rate model.

FIG. 4A depicts a time-domain signal from a heart rate sensor for use in step 301 of FIG. 3A.

FIG. 4B depicts a spectrum 410 of the time-domain signal of FIG. 4A for use in step 302 of FIG. 3A.

FIG. 4C depicts an example probability density function 420 which has the widest possible spread, consistent with step 312 of FIG. 3A.

FIG. 4D depicts example probability density functions 430, 431 and 432 which have different spreads sp1, sp2 and sp3, respectively, consistent with step 312 of FIG. 3A.

FIG. 4E is an example of a uniform distribution of a PDF, consistent with step 312 of FIG. 3A.

FIG. 4F depicts an example probability density function 440 which has a spread of sp1=fu1−fl1, a center value of cv1 and a peak amplitude of Pmax, consistent with step 312 of FIG. 3A.

FIG. 4G depicts an example spectrum 450 of a time-domain signal from a heart rate sensor, consistent with step 302 of FIG. 3A.

FIG. 4H depicts an example spectrum 460 of a time-domain signal from a heart rate sensor, consistent with step 302 of FIG. 3A.

FIG. 4I depicts an example spectrum 470 which corresponds to the spectrum 460 of FIG. 4G, after processing consistent with step 306 of FIG. 3A.

FIG. 5A depicts a plot 500 of an example calorie burn rate, consistent with step 307 of FIG. 3A.

FIG. 5B depicts a plot 510 of an example motion-based heart rate corresponding to the calorie burn rate of FIG. 5A, consistent with step 309 of FIG. 3A.

FIG. 6A depicts a relationship between activity type and calorie burn rate (CBR) consistent with step 321 of FIG. 3B.

FIG. 6B depicts a relationship between heart rate and calorie burn rate (CBR) consistent with step 309 of FIG. 3A.

FIG. 6C depicts example accelerometer readings during an activity, consistent with step 320 of FIG. 3C.

### DETAILED DESCRIPTION

Devices and techniques are provided herein which provide a heart rate measurement of a user with increased reliability. A best estimate of the current heart rate is made based on a heart rate model and a current heart rate value from a heart rate sensor. The techniques may be embodied in an activity monitor, for instance. An activity monitor is worn by a user, such as on the wrist, and includes circuitry for detecting heart rate and an energy expenditure, e.g., calories burned. A activity monitor can be a dedicated device or can be incorporated into another device such as a cell phone or other computing device.

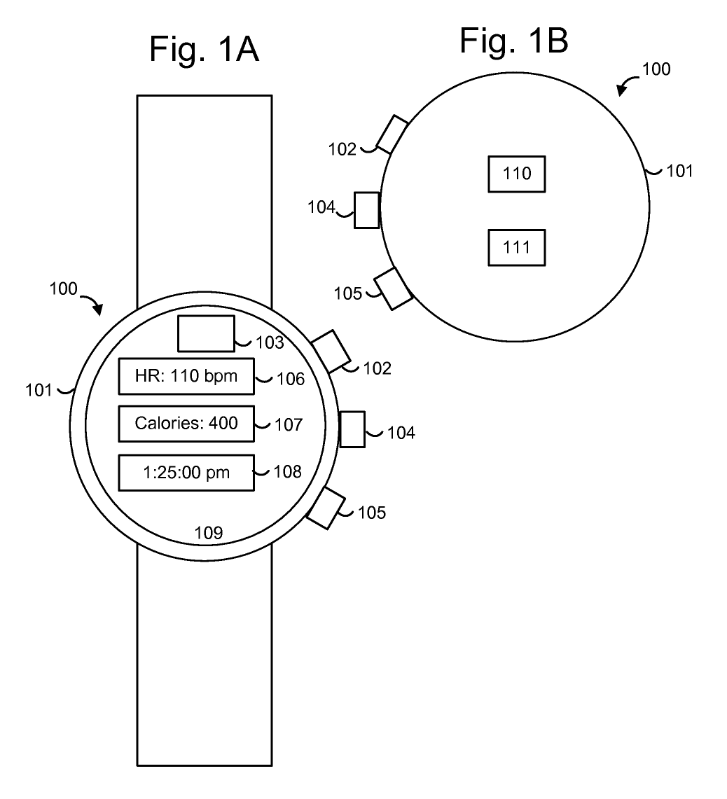

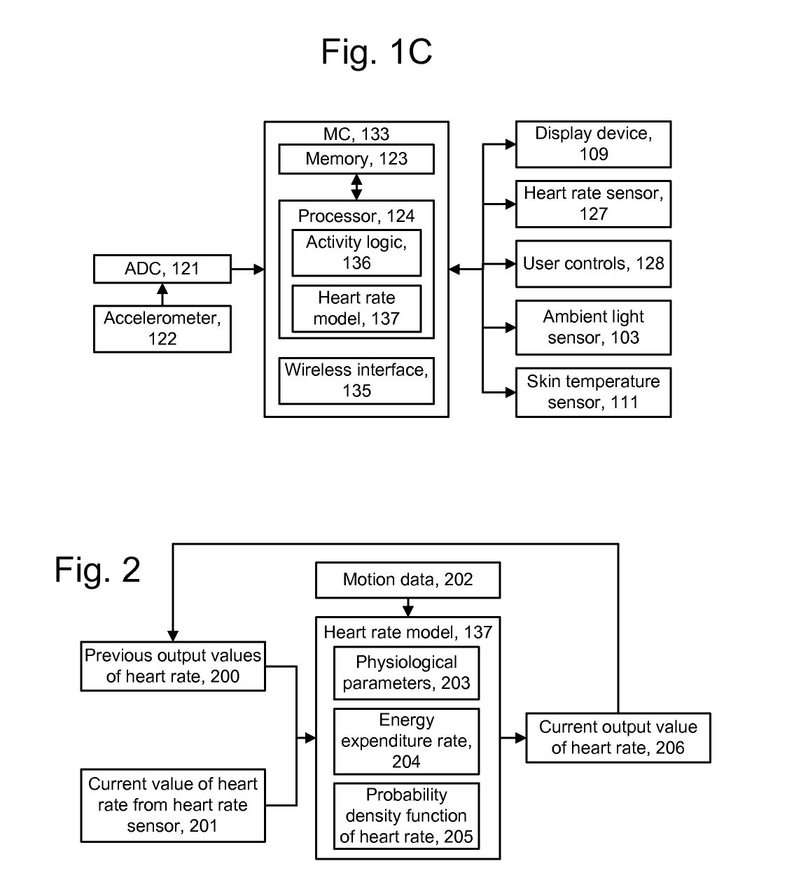

FIG. 1A depicts a front view of an example activity monitor. The activity monitor 100 can be a wristwatch type device comprising a watch face and a strap for wearing around the wrist in this example, but other implementations are possible. For example, such monitors can be worn on the belt, head, chest, arm or carried in the pocket. A monitor could also include multiple components which are attached to different parts of the body. For example, the different components can include accelerometers which are attached to different parts of the body, e.g., the arm, leg or foot, to gain a more complete understanding of the user's activity, including posture. The activity monitor 100 includes a case 101, a crown 104, a mode select button 105 and an exercise mode button 102. A display device 109 includes an ambient light sensor 103, a region 106 which depicts a heart rate (HR) (e.g., 110 beats per minutes or bpm), a region 107 which depicts an amount of calories (e.g., 400 calories) consumed in a time period such as in the current day, and a region 108 which depicts a time of day (e.g., 1:25:00 pm). The mode select button 105 may allow the user to activate different operational modes and to input user-specific physiological parameters such as age, gender, height, weight, body mass index or maximum rate of oxygen consumption (VO2max).

The activity monitor can include a heart rate sensor which automatically determines the heart rate continuously, periodically or at other specified times as determined by a control or based on a manual user action. For example, the user may touch the exercise mode button 102 to indicate that a workout session is beginning in which case continuous heart rate measurements are desired. Or, in a free living application, the heart rate is determined automatically during periods of interest, such as when significant amount of activity is detected.

The heart rate sensor can be based on the use of ultrasonic, optical or electrical signals, for instance. For a wrist worn device, it is convenient to use ultrasonic or optical transmitters and receivers on the back of the device. These types of monitors are popular since they do not require an electrode-carrying chest strap. On the other hand, an example of the use of electrical signals is provided by an ECG-based monitor which is worn on a chest strap, where the electrodes of the monitor are constantly in contact with the body and can therefore continuously determine heart rate. Heart rate data can be transmitted from the chest strap to the display device 109 for viewing by the user.

FIG. 1B depicts a rear view of the activity monitor of FIG. 1A. In this example, an optical component 110 emits light into the user's body and detects reflections, to determine the heart rate. A skin temperature sensor 111 is also provided. Skin temperature can be used to determine activity and calories burned, for instance.

FIG. 1C depicts an example block diagram of the activity monitor of FIG. 1A. A micro-controller (microprocessor controller, MC) 133 includes a processor 124 having activity analysis logic 136 and a heart rate model 137. The processor communicates with a memory 123 and a wireless interface 135. The wireless interface may communicate wirelessly with another computing device. The computing device may have a user interface which displays information from the activity monitor and allows the user to enter information such as physiological parameters and configure settings of the activity monitor. For example, the activity monitor may communicate with a cell phone via a piconet such as using the BLUETOOTH™ protocol.

The micro-controller communicates with a number of components including an analog-to-digital converter (ADC) 121, the display device 109, a heart rate sensor 127, user controls 128, the ambient light sensor 103 and the skin temperature sensor 111.

Also provided is an accelerometer 122 (an example of a motion sensor), such as a three-axis accelerometer. The accelerometer may provide an analog output signal representing acceleration in one or more directions. For example, the accelerometer can provide a measure of acceleration (g-forces) with respect to x, y and z axes. The analog outputs are digitized by the ADC 121 and digital samples (motion data) are provided to the processor 124. The ADC could be part of the MC or processor. The accelerometer 122 provides acceleration readings at a prescribed rate such as multiple times per second. The processor can continuously or periodically process samples from the accelerometer. The acceleration samples can be used to determine an activity level of the user and, in some cases, a type of the activity. Based on this, an energy expenditure rate and other metrics can be calculated. Another example of a motion sensor which may be used is a gyrometer, which provides a measure of angular velocity with respect to x, y and z axes. Another example of a motion sensor which may be used is an inclinometer, which provides a measure of pitch, roll and yaw that correspond to rotation angles around x, y and z axes.

The display device may be used to display information such as a current value of a heart rate, an energy expenditure rate (calorie burn rate) and a cumulative energy expenditure (total calories burned).

The heart rate sensor may be an optical sensor, for example. The heart rate sensor determines a current heart rate of a user either on demand or continuously.

The user controls may be buttons on the activity monitor which allow the user to enter commands such as to activate the display or configure the activity monitor. The user controls 128 can include the mode select button 105 of FIG. 1A, for instance and associated components.

The ambient light sensor may include, e.g., a light-dependent resistor or a photodiode and can be used to determine information such as whether the user is in a dark room and therefore is likely sleeping. A light sensor can provide an ambient light reading as a lux value. The skin temperature sensor may include, e.g., a thermistor, a type of resistor whose resistance varies with temperature, and can be used to determine information such as whether the user is sleeping or exercising.

The diagram is meant to provide a high level understanding of the activity monitor. Specific implementations can take many forms. For example, accelerometer signals can be subject to analog signal processing, analog to digital conversion, time domain processing, conversion to the frequency domain such using a Fast Fourier Transform and frequency domain processing. Heart rate signals can be similarly processed.

The MC may be in communication with each of the other components and transmit signals to them and/or receive signals from them. The memory 123 can store code which is executed by the processor to perform the functionality described herein. The memory is an example of a tangible computer-readable storage apparatus or memory having computer-readable software embodied thereon for programming a processor to perform a method. For example, non-volatile memory can be used. Volatile memory such as a working memory of the processor can also be used. The computer-readable storage apparatus may be non-transitory and exclude a propagating signal.

FIG. 2 depicts a software block diagram of an implementation of the heart rate model 137 of FIG. 1C. The heart rate model 137 determines a current heart rate of the user based on factors such as previous output values of the heart rate 200 and motion data 202. The heart rate determined by the model is then compared to a current value of heart rate from a heart rate sensor 201 as a check on the validity of the value of the heart rate sensor. If the value from the heart rate sensor appears to be correct, it is output as the current value of the heart rate, e.g., to a display device and/or for storage in a memory. If the current value from a heart rate sensor does not appear to be correct, the heart rate determined by the model is output in place of the current value from the heart rate sensor, in one approach. Another approach is to output a value that is based on both the heart rate determined by the model and the current value of heart rate from a heart rate sensor.

The heart rate model stores physiological parameters 203 of the user to tailor its heart rate estimate to the user. At block 204, an energy expenditure rate such as calorie burn rate can be determined based on the motion data and the physiological parameters, for instance. In one approach, the heart rate estimate of the model can be provided in the form of a probability density function 205 which has a spread and a center value. Further details are provided below. At block 206, the model provides a current output value of the heart rate.

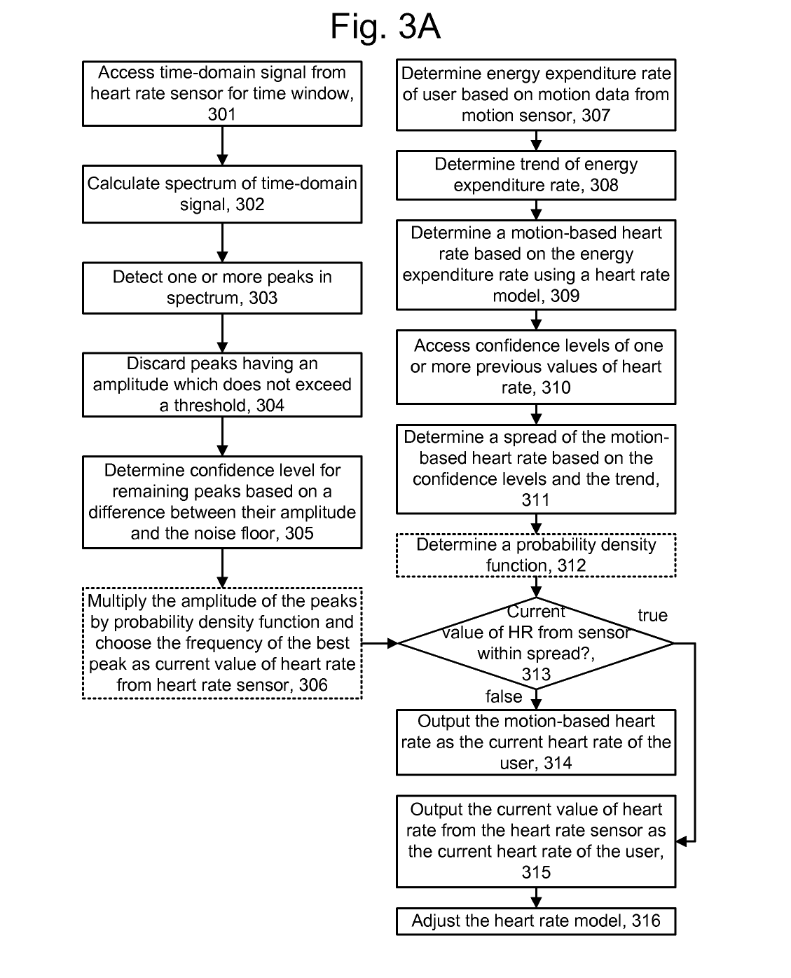

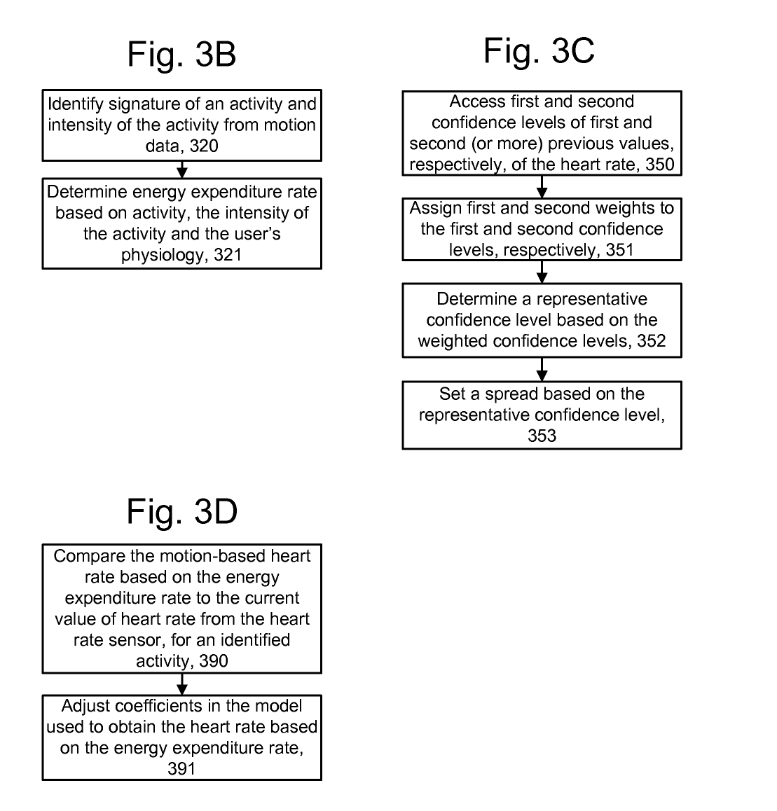

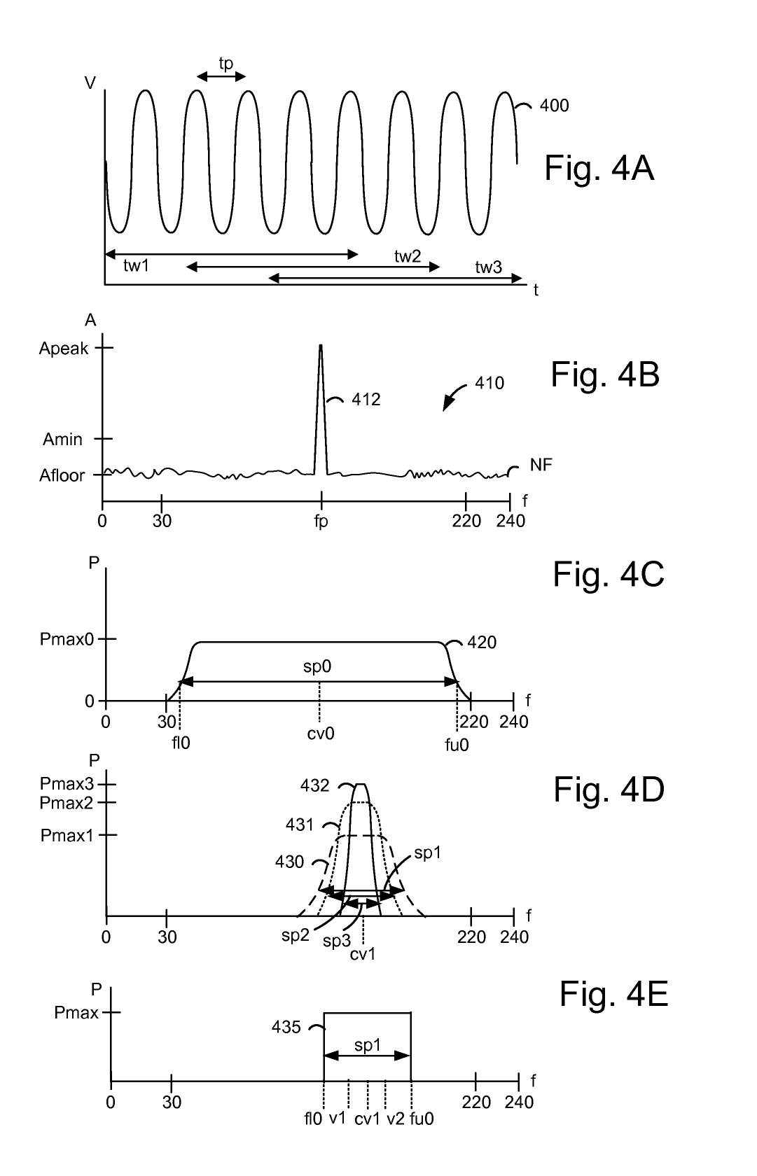

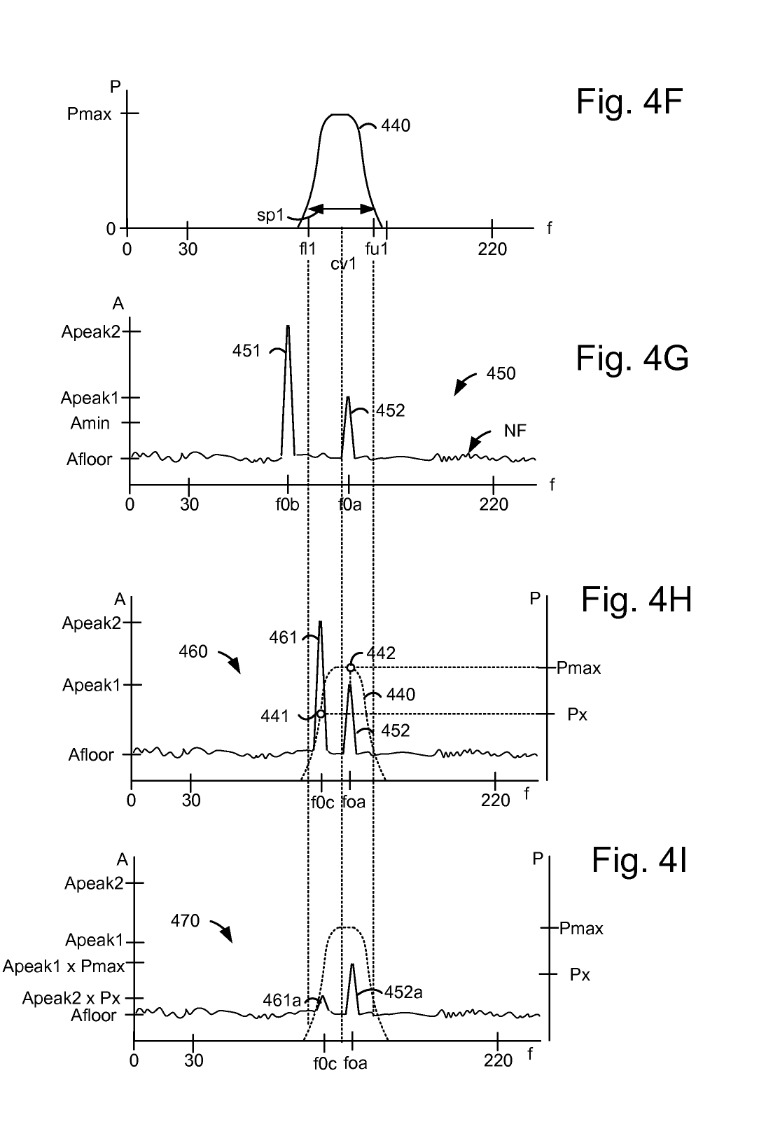

FIG. 3A depicts a flowchart of a process for determining heart rate using the heart rate model 137 of FIG. 2. Steps 301-306 and steps 307-316 may be performed concurrently, in one approach. Step 301 involves accessing a time-domain signal from a heart rate sensor for a time window, such as the past few seconds. See, e.g., FIG. 4A for further details. Step 302 involves calculating a spectrum (frequency-domain signal) of the time-domain signal such as by using the Fast Fourier Transform (FFT). See, e.g., FIGS. 4B, 4G and 4H for further details. Step 303 involves detecting one or more peaks in the spectrum. Step 304 involves discarding peaks having an amplitude which does not exceed a threshold. See, e.g., FIG. 4G for further details. Step 305 involves determining a confidence level for remaining peaks based on a difference between their amplitude and the noise floor. Optional step 306 includes multiplying the amplitude of the peaks by a probability density function and choosing the frequency of the best peak as the current value of the heart rate from the heart rate sensor. See, e.g., FIGS. 4H and 4I for further details.

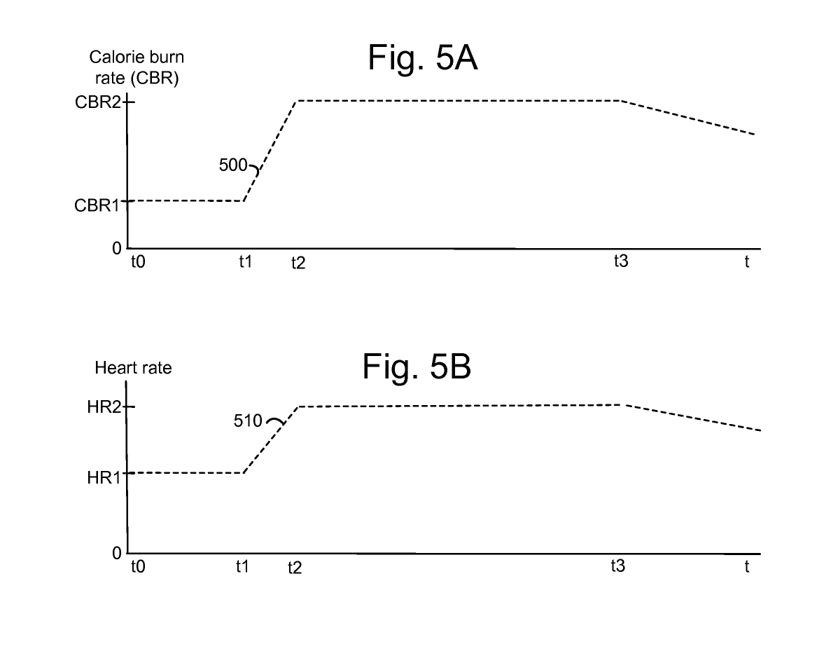

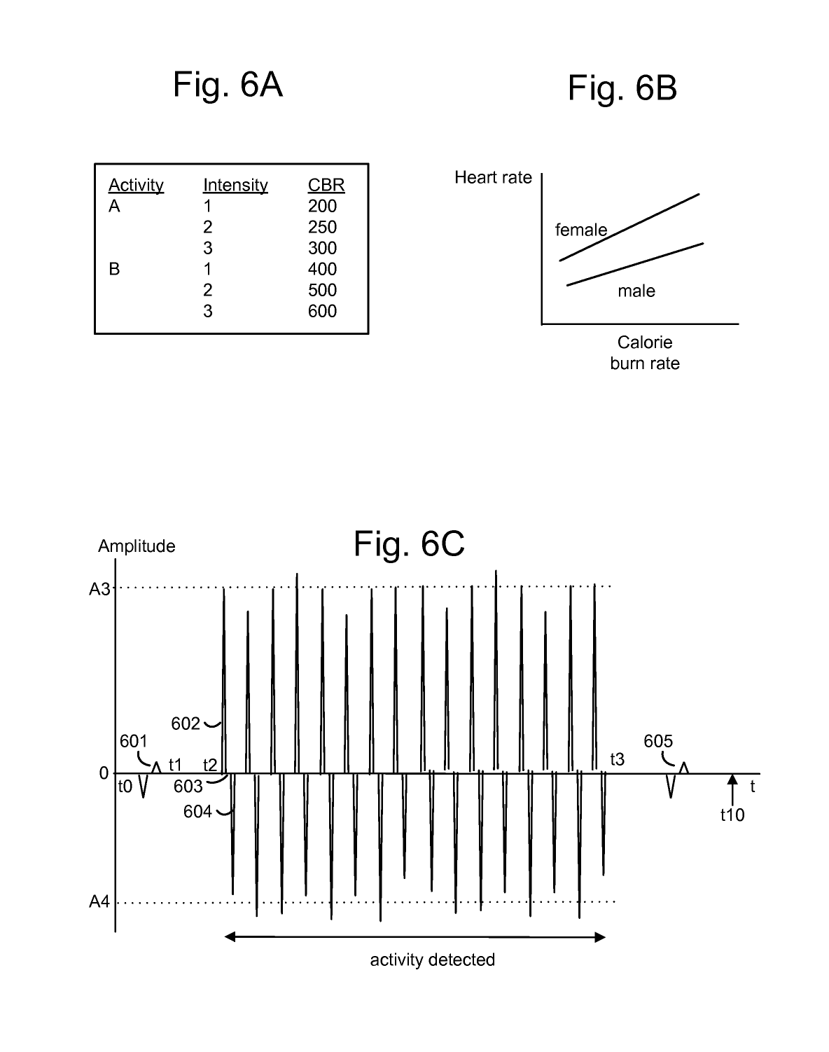

Step 307 includes determining an energy expenditure rate of the user based on motion data from the motion sensor. This can involve determining a type of activity the user is performing See, e.g., FIGS. 6A and 6C for further details. Step 308 includes determining a trend of the energy expenditure rate, e.g., steady, increasing or decreasing. This step can involve accessing energy expenditure rate data from memory over a recent time period and determining a trend of the energy expenditure rate (e.g., steady, increasing or decreasing), based on the data. The energy expenditure rate can be stored for a time period such as several seconds and a trend of the rate can be determined, such as from a slope of the rate. For example, the trend can be increasing when the slope exceeds a minimum positive, non-zero slope, decreasing when the slope is below a minimum, negative non-zero slope, or steady when the slope is below the minimum positive, non-zero slope and above the minimum, negative non-zero slope. For example, the trend can be the plot in FIG. 5A.

Step 309 involves determining a motion based-heart rate, e.g., a heart rate that is based on the energy expenditure rate, using he heart rate model. A model can use an equation to obtain heart rate based on energy expenditure rate, in one example. Other approaches could use multiple equations such as in a set of linear equations, or use a state machine, a neural network and so forth. In one approach, different models of the heart rate are used, and a heart rate is obtained from one of them or from a combination or two or more of them.

Various studies provide a correspondence between heart rate and energy expenditure rate. One study provides an energy expenditure rate as a function of gender, age and weight. For women, the equation is: CBR=−20.4022+0.4472×heart rate−0.1263×weight+0.074×age. For men, the equation is: CBR=−55.0969+0.6309×heart rate+0.1988×weight+0.2017×age. This equation can be re-written as: heart rate=(CBR+55.0969−0.1988×weight−0.2017×age)/0.6309. The form of this equation is: heart rate=c1+c2×CBR (energy expenditure rate)+c3×parameter (e.g., weight)+c4×parameter (e.g., age) . . . , where c1-c4 are coefficients for the CBR and the physiological parameters. The CBR above is in units of kJ/min, where 1 food calorie=4.2 kJ. By inserting a value for CBR in the above equation, a motion-based heart rate can be determined.

Step 310 involves accessing one or more previous values of heart rate and confidence levels, if available, from memory. These can be previous values which are output from the model and can be pass through values from the heart rate sensor and/or motion-based heart rate values calculated by the model. In one approach, the confidence level is available for the values from the heart rate sensor but not for the values calculated by the model. In another approach, a confidence level of a value calculated by the model is provided by an associated spread.

Step 311 determines a spread of the motion-based heart rate based on the confidence levels of the one or more previous values of heart rate and the trend of the energy expenditure rate. The motion-based heart rate can be a center value of the spread. The spread indicates a range around the motion-based heart rate in which the actual heart rate is expected. The spread is an indication of a confidence level or an uncertainty of the motion-based heart rate. A higher spread indicates a higher uncertainty. In one approach, this step involves selecting a spread from among a plurality of predetermined spreads. See, e.g., FIG. 4D for further details of different spreads.

Further, in one approach, when the trend of the energy expenditure rate is increasing or decreasing, the spread of the PDF can be increased relative to the case where the trend is steady. In another approach, the spread of the PDF is increased by different amounts for an increasing or decreasing trend. In another approach, the trend can include a degree of increasing or decreasing, and the spread of the PDF can be increased by a relatively large amount when the degree of increasing or decreasing is relatively larger. The increase in the PDF reflects an increased uncertainty in the heart rate when the heart rate is changing. See also, e.g., FIGS. 4E and 5A for further details.

Optional step 312 determines a probability density function (PDF) of a heart rate from the model. The PDF may have a dispersion metric which is the spread. Further, the PDF may have a central value or central tendency which is a center of the spread. This central value may be the motion-based heart rate. The PDF can have a uniform shape in which the value of the PDF is either 0 or 1 (e.g., FIG. 4E), or it can have a non-uniform shape. One example of a non-uniform shape is a shape with a peak and sides which slope downward from the peak. The downward slope can be symmetric with respect to the peak. See, e.g., FIG. 4D for further details.

The inputs to the model which are used to determine the PDF can include previous values of heart rate and associated confidence levels, energy expenditure rate (e.g., a current rate), and a trend of the energy expenditure rate. The PDF can be based on one or more previous values of heart rate by virtue of the model learning a correlation between energy consumption rate and heart rate based on the previous values of heart rate from the heart rate sensor.

Decision step 313 determines if the current value of the heart rate (HR) from the heart rate sensor is within the spread. If this is true, step 315 outputs the current value of the heart rate from the heart rate sensor as the current heart rate of the user. In other words, the current value of the heart rate from the heart rate sensor is within the expected range according to the model and is therefore passed through to the output. However, if decision step 313 is false, step 314 outputs the motion-based heart rate as the current heart rate of the user. In other words, the current value of the heart rate from the heart rate sensor is not within the expected range according to the model, so the model outputs a value it has calculated and believes is more correct. Or, the output heart rate can be based on both the current value of the heart rate from the heart rate sensor and the motion-based heart rate, e.g., as an average of the two, or some other value between the two.

Step 316 involves adjusting the heart rate model. See, e.g., FIG. 3D for further details. This can involve a machine learning process, for example.

FIG. 3B depicts a flowchart of a process for use in step 307 of FIG. 3A for determining an energy expenditure rate of a user based on motion data from a motion sensor. Step 320 includes identifying a signature of an activity and an intensity of the activity from motion data. Generally, the motion sensor can detect different predetermined activities. One approach involves identifying a signature of a specific exercise. For example, in a test process, a motion sensor can be worn by a population of users who perform specific exercises and the corresponding accelerometer readings are recorded. This could be done as part of the development of the activity monitor by the manufacturer. The population can represent users with different physiological parameters. A given exercise can be performed with different levels of intensity as well. For example, for running, the intensity can be based on the speed of the user. The speed can be determined from the step rate and an estimated stride, where the stride can be based on factors such as the user's height.

Subsequently, when the end user performs a given exercise, the exercise is identified according to a signature based on the physiological parameters of the end user. In one approach, the activity monitor is set up by the end user to recognize certain activities. See also, e.g., FIG. 6C for further details.

Step 321 includes determining an energy expenditure rate based on the activity, the intensity of the activity and the user's physiology. Generally, an energy expenditure, in terms of food calories (kcal) per minute (a calorie burn rate or CBR), can be associated with each activity and intensity. Moreover, the energy expenditure rate can be adjusted based on the user's physiological parameters. For a given activity, the energy expenditure rate is higher when the intensity is higher. Also, the energy expenditure rate is higher when the user's weight is higher. The energy expenditure rate is strongly dependent on weight. For example, for the activity of running at 5 mph, the energy expenditure rate is 472, 563, 654 or 745 calories per hour based on a user weight of 130, 155, 180 or 205 pounds, respectively. For the same activity but with a higher intensity of running at 6 mph, the energy expenditure rate is 590, 704, 817 or 931 calories per hour based on a user weight of 130, 155, 180 or 205 pounds, respectively. In some cases, the user is resting, such that a basal metabolic rate (BMR) or a resting metabolic rate (RMR) applies. The BMR applies to a user who has just awoke after sleeping while the RMR applies when the user is awake but resting. BMR and RMR are a function of weight, height and age.

FIG. 3C depicts a flowchart of a process for use in step 311 of FIG. 3A for determining a spread based on confidence levels. Generally, the spread can be based on confidence levels of one or more previous values of heart rate, such as from the heart rate sensor. These one or more previous values can from consecutive values immediately preceding the current value of the heart rate sensor, for example. Generally, the spread is inversely proportional to the confidence level—a higher confidence level results in a smaller spread. Step 350 includes accessing first and second (or more) confidence levels of first and second previous values, respectively, of the heart rate. The second value is before the first value. The first value can be immediately before the current value, and the second value can be immediately before the first value. Other approaches are possible as well. Step 351 involves assigning first and second weights to the first and second confidence levels, respectively. The first weight can be higher than the second weight based on the theory that the confidence level of the more recent heart rate value is more predictive of the accuracy of the current heart rate value. Step 352 involves determining a representative confidence level based on the multiple weighted confidence levels. Step 353 involves determining a spread based on the representative confidence level.

For example, assume a confidence level scale of 0 to 1, where 1 is most confident. Also, assume the first previous value has a confidence level of 0.8 and a weight of 0.6, and the second previous value has a confidence level of 0.7 and a weight of 0.4. A representative confidence level may then be 0.8×0.6+0.7×0.4=0.48+0.28=0.76. The sum of the weights is one in this example (0.6+0.4). A scale may be provided in which the spread is a function of the representative confidence level, e.g., sp0, sp1, sp2 or sp3 for confidence levels of 0.0-0.7, 0.7-0.8, 0.8-0.9 and 0.9-1.0, respectively. See examples of sp, sp1, sp2 or sp3 in FIGS. 4C and 4D.

In another example, only one confidence level of a prior value is used, such as the confidence level of the most recent prior value. In another example, the process involves determining a number N of consecutive values among a plurality of previous values for which the respective confidence level exceeds a threshold, where the spread is inversely proportional to the number N. For instance, if there are N=10 consecutive previous heart rate values from the heart rate sensor with a relatively high confidence, e.g., above 0.8, the spread can be made relatively narrow. On the other hand, if there are no more than N=3 consecutive previous heart rate values from the heart rate sensor with a relatively high confidence, the spread can be made relatively wide.

FIG. 3D depicts a flowchart of a process for use in step 316 of FIG. 3A for adjusting a heart rate model. Step 390 compares the motion-based heart rate based on the energy expenditure rate to the current value of heart rate from the heart rate sensor, for an identified activity. Step 391 adjusts coefficients in the model used to obtain the heart rate based on the energy expenditure rate.

Even though the equation used to determine the motion-based heart rate can consider physiological parameters, the equation is for an average user with a given set of physiological parameters. Moreover, other factors can affect energy expenditure, such as ambient temperature and humidity. Additionally, the detection of an activity by a user is subject to uncertainty. The motion-based heart rate can thus differ from the actual heart rate. The model can be periodically adjusted based on information such as a high-confidence current value from the heart rate sensor, in an attempt to provide results which more closely match the heart rate sensor, for a specific user.

An equation above provided a correspondence between heart rate and CBR as follows: heart rate=c1+c2×CBR (energy expenditure rate)+c3×parameter (e.g., weight)+c4×parameter (e.g., age), where c1-c4 are coefficients for the CBR and the physiological parameters. In this case, the adjusting of the model can involve adjusting one or more of the coefficients. In a learning process of the model, the coefficients can be adjusted to provide a more accurate correspondence for a specific user. For example, when the equation indicates that heart rate based on the CBR should be 120 bpm, but the heart rate based on a reading from the heart rate sensor is 110 bpm, one or more of the coefficients can be increased. Over time, in an iterative process, the heart rate determined from the motion more closely matches the heart rate determined by the heart rate sensor. The model therefore becomes a valuable tool in checking the validity of readings from the heart rate sensor, particularly at times when the readings from the heart rate sensor have a low confidence or are not available at all. This can occur, e.g., when the activity monitor and the heart rate sensor are moving relative to the body or are otherwise not in good contact with the user's body. This might be caused by a wrist-worn activity monitor being worn too loosely, slippage due to sweat or lotion on the skin, or other factors.

FIG. 4A depicts a time-domain signal 400 from a heart rate sensor for use in step 301 of FIG. 3A. One example of heart rate sensor injects light into a user's body such as from the back of a wrist worn activity monitor, and senses reflections of the light from the body. The amplitude of the reflections can be depicted by a time varying voltage which varies with the movement of blood vessels in the body. This movement can involve the periodic expansion and contraction of blood vessels at the frequency of the heart rate, for instance. In this example, the time-domain signal is plotted using voltage on the vertical axis and time on the horizontal axis. The signal may be generally sinusoidal with periodic peaks. An example period between the peaks is tp. The period, which is the inverse of the heart rate, can vary over time depending on what the user's activity. Although, over a few seconds, the heart rate may be fairly steady.

In calculating the spectrum of the time-domain signal, one approach is to transform a portion or window of the signal. For each new reading, the window is moved and the transform is based on the portion of the signal in the current window. The duration of a window should be sufficient to capture the frequency characteristic by encompassing two or more peaks at the lowest expected heart rate, corresponding to the longest heartbeat period. For example, if the lowest expected heart rate is 30 beats per minute (bpm), corresponding to a period of 2 seconds, the window should be at least 2 seconds. In practice, the window can be longer, such as 5-6 seconds, to accurately capture the heart beat period. If the window is too long, the current value of the heart rate will be averaged out with previous values, and the computational cost increases. The spectrum obtained from each window results in a reading of the heart rate. Each window can overlap by, e.g., 1-2 seconds so that a new heart rate value is obtained every 1-2 seconds. Example windows tw1, tw2 and tw3 are depicted.

FIG. 4B depicts a spectrum 410 of the time-domain signal of FIG. 4A for use in step 302 of FIG. 3A. The spectrum can be obtained using a Discrete Fourier transform (DFT) such as the FFT. The spectrum is plotted using an amplitude on the vertical axis (which may be a logarithmic scale) and frequency (e.g., heart rate) on the horizontal axis. The spectrum can be an amplitude spectrum or power spectrum, for instance. The single-sided power spectrum of a voltage waveform is in units of Volts rms squared. The amplitude spectrum is obtained by taking the square root of the power spectrum. In this example, heart rates between 30 and 220 bpm are considered to be valid for the general human population. However, in situations in which the resting heart rate and the maximum heart rate for a particular user are known, this range can be used instead. For example, the resting heart rate of a user may be 70 bpm and the maximum heart rate may be 190 bpm, so that the allowed range is (70−margin) to (190+margin), where a margin of error is added such as 5-10 bpm. The noise floor NF represents the lowest possible amplitude (Afloor) of the spectrum. The theoretical noise floor of the FFT is equal to the theoretical signal to noise ratio plus the FFT process gain, 10×log(M/2), where M is the size of the FFT.

Here, there is a single distinct peak 412 at a frequency of fp with an amplitude of Apeak. The peak is not discarded because its amplitude is above a minimum threshold of Amin, consistent with step 304 of FIG. 3A. A confidence level of the peak can be determined as Apeak-Afloor (e.g., decibels or Volts rms squared), for instance, consistent with step 305 of FIG. 3A, or some other function proportional to the amount by which Apeak exceeds Afloor.

FIG. 4C depicts an example probability density function 420 which has the widest possible spread, consistent with step 312 of FIG. 3A. A probability density function PDF(HR) is, e.g., a function of a continuous random variable (e.g., heart rate or HR) that describes the relative likelihood or probability for the random variable to take on a given value. The likelihood of the heart rate being a particular value is given by the height of the probability density function at that value, e.g., by evaluating PDF(HR) at a certain HR. Similarly, the probability of the heart rate falling within a particular range of values is given by the integral of the probability density function over the range. The area under the PDF sums to one. A PDF can be a continuous plot or can be comprised of a number of bins for different ranges of heart rate. A PDF could alternatively be represented by a cumulative distribution function, where the PDF is the derivative of the cumulative distribution function.

The PDF is plotted using a likelihood or probability P on the vertical axis and frequency (e.g., heart rate) on the horizontal axis. The PDF may have various shapes and spreads. One example shape is flat for a majority of the non-zero values. Once example is the Tukey window, also known as the tapered cosine window, which has a flat top with tapered edges. Gaussian and uniform shapes are other examples. Moreover, the spread can vary at different times based on the reliability of the heart rate estimate of the model. A narrower spread corresponds to a more reliable heart rate estimate. The spread can be a dispersion metric such as variance, standard deviation, interquartile range or other measure of dispersion.

Assume the activity monitor has just been attached to the wrist of the user, so there is no information available to the model about a previous heart rate or energy expenditure rate. In this example, the PDF is zero for frequencies less than 30 bpm and greater than 220 bpm, and rises up to a maximum probability of Pmax0. The spread sp0 is from a little more than 30 bpm (e.g., about 40 bpm. a lower bound frequency fl0 of the spread) to a little less than 220 bpm (e.g., about 210 bpm, an upper bound frequency fu0 of the spread). A motion-based heart rate may be a center value cv0 of the spread (e.g., 40+170/2=125 bpm) and represents the model's best estimate of the current heart rate. In another option, consistent with step 311 of FIG. 3, sp0 and cv0 are determined without determining the PDF.

FIG. 4D depicts example probability density functions 430, 431 and 432 which have different spreads sp1, sp2 and sp3, respectively, consistent with step 312 of FIG. 3A. The PDFs have the same general shape and are aligned with one another, but have progressively smaller spreads. Once the model obtains additional information such as an energy expenditure rate and previous values of the heart rate (e.g., from the heart rate sensor), the model can provide a more accurate PDF of the heart rate. In this example, the model provides a PDF 430 which has a spread sp1 with a center value of cv1, then a PDF 431 which has a spread sp2 also with the center value of cv1, then a PDF 432 which has a spread sp3 with the center value of cv1. Or, the center value could be modified slightly with each new PDF. The height of each successive PDF increases, from Pmax1 to Pmax2 to Pmax3 in this example as the heart rate estimate from the model becomes more accurate. Generally, the PDF will become relatively narrow when the heart rate is at a fixed level and relatively wide when the heart rate is changing rapidly. The PDF will also become relatively narrow when the heart rate values from the heart rate sensor have a relatively high confidence and relatively wide when the heart rate values from the heart rate sensor have a relatively low confidence. The PDF will also become relatively narrow when an energy expenditure rate from the motion sensor is at a fixed level and relatively wide when the energy expenditure rate is changing rapidly.

The PDFs 420 and 430-432 are examples of a plurality of predetermined non-uniform PDFs with predetermined spreads.

FIG. 4E is an example of a uniform distribution of a PDF, consistent with step 312 of FIG. 3A. The uniform distribution 435 has a fixed likelihood (Pmax) between a lower frequency fl0 and an upper frequency fu0. The spread is sp1, as in FIG. 4D. The area under the PDF sums to one such that Pmax×sp1=1. A center value of cv1 may represent the motion-based heart rate as discussed in connection with step 309 of FIG. 3A. In another possibility, the motion-based heart rate is not at the center of the spread. For example, if there is a decreasing trend of the motion-based heart rate, the motion-based heart rate may be at an upper portion of the spread, such as at the value v2. This approach recognizes that the heart rate is decreasing so the spread should encompass a wider range below the motion-based heart rate (e.g., the range v2-fl0) than a range above the motion-based heart rate (e.g., the range v2-fl0). Or, if there is an increasing trend of the motion-based heart rate, the motion-based heart rate may be at a lower portion of the spread, such as at the value v1. This recognizes that the heart rate is increasing so the spread should encompass a wider range above the motion-based heart rate (e.g., the range fu0-v1) than a range below the motion-based heart rate (e.g., the range v1-fl0).

In another approach, the spread is relatively wider when the trend is increasing or decreasing and relatively narrower when the trend is steady. Combinations of the above approaches may be used as well.

FIG. 4F depicts an example probability density function 440 which has a spread of sp1=fu1−fl1, a center value of cv1 and a peak amplitude of Pmax, consistent with step 312 of FIG. 3A. Assume this is a current PDF of the model. FIG. 4F-4I have an aligned frequency scale on their horizontal axes.

FIG. 4G depicts an example spectrum 450 of a time-domain signal from a heart rate sensor, consistent with step 302 of FIG. 3A. A first highest peak 451 is outside the spread sp1 of the PDF 440 of FIG. 4F and a second highest peak 452 is within the spread. In some cases, multiple strong peaks are present in the heart rate spectrum. Typically, one of the peaks represents the actual heart rate and the other peaks are present as noise. One source of noise is motion of the activity monitor. For example, the spectrum may have a peak consistent with a periodic movement of the activity monitor. In this case, the peak 451 of amplitude Apeak2 which is outside the spread is determined to be incorrect while the peak 452 of amplitude Apeak1<Apeak2 within the spread is assumed to be correct. Thus, the lower amplitude peak is correct in this example. In contrast, selection of the strongest peak without reference to a PDF or spread would result in an incorrect heart rate from the heart rate sensor. The peak 452 may therefore be selected as the current value of the heart rate from the heart rate sensor. Moreover, in some cases, the noise-based peak can be close to the actual heart rate and within the spread of the PDF. In such situations, the shape of the PDF can be used to discount the noise-based peaks, as described below.

In one approach, a peak having an amplitude which does not exceed a threshold (e.g., Amin) is discarded and not considered as a potential heart rate, consistent with step 304 of FIG. 3A.

FIG. 4H depicts an example spectrum 460 of a time-domain signal from a heart rate sensor, consistent with step 302 of FIG. 3A, where a first highest peak 461 of amplitude Apeak2 and the second highest peak 452 of amplitude Apeak1 are within the spread sp1 of the PDF 440 of FIG. 4F. The left hand scale represents the amplitude of the spectral peaks and the right hand scale represents the amplitude of the PDF. The PDF has a shape with a maximum value at the frequency or heart rate of cv1 in this example, and lower values corresponding to the sloping sides of the PDF. A peak which is frequency-aligned with the lower values of the PDF can be attenuated such as by multiplying the amplitude of the peak with a value of the PDF evaluated at the frequency of the peak. For example, the peak 461 is at a frequency of f0c. The PDF evaluated at f0c has a value of Px as represented by point 441 on the plot of the PDF. Similarly, the peak 452 is at a frequency of f0a, where f0c<cv1<f0a. The PDF evaluated at f0a has a value of Pmax as represented by point 442 on the plot of the PDF. Pmax>Px>0.

FIG. 4I depicts an example spectrum 470 which corresponds to the spectrum 460 of FIG. 4G in which the amplitudes of the peaks 452 and 461 are multiplied by corresponding values Pmax and Px, respectively, of the PDF 440 to provide adjusted peaks 452a and 461a, respectively, consistent with step 306 of FIG. 3A. The peak 461 of amplitude Apeak2 has been reduced to the peak 461a of amplitude Apeak2×Px, while the peak 452 of amplitude Apeak1 has been reduced to the peak 452a of amplitude Apeak1×Pmax (>Apeak2×Px). In this example, the shape of the PDF is used to identify a best estimate of the current heart rate from the heart rate sensor. This is another example where selection of the strongest peak without reference to a PDF or spread would result in an incorrect heart rate from the heart rate sensor. This approach resolves a scenario in which there are multiple apparently valid peaks which are close together in frequency.

This example involves obtaining a spectrum 460 of a time-domain signal 400 from the heart rate sensor 127, determining a magnitude (Apeak2) of a first highest peak 461 and a magnitude (Apeak1) of a second highest peak 452 in the spectrum, evaluating the non-uniform probability density function 440 at a frequency f0c of the first highest peak to provide a first likelihood (Px), and obtaining a first adjusted value (Apeak2×Px) which is based on multiplying the magnitude of the first highest peak by the first likelihood, evaluating the non-uniform probability density function at a frequency of ma the second highest peak to provide a second likelihood (Pmax), and obtaining a second adjusted value (APeak1×Pmax) which is based on multiplying the magnitude of the second highest peak by the second likelihood, and using the frequency of the first highest peak as the current value from the heart rate sensor if the first adjusted value is greater than the second adjusted value, and using the frequency of the second highest peak as the current value from the heart rate sensor if the second adjusted value is greater than the first adjusted value (as in this example).

FIG. 5A depicts a plot 500 of an example calorie burn rate, consistent with step 307 of FIG. 3A. The horizontal axis is for time and the vertical axis is for calorie burn rate. The plot indicates that the calorie burn rate is CBR1 from t0-t1, increases to CBR2 from t1-t2, remains relatively steady at CBR2 from t2-t3 and begins to decrease starting at t3. For example, the user may be at rest from t0-t1, participate in an exercise session from t1-t3, then have a cool off period beginning at t3.

The trend of the energy expenditure rate can be the slope of the plot of CBR, in one approach. Thus, the slope of the CBR is zero from t0-t1 and t2-t3, indicating a steady CBR. The slope of the CBR is positive from t1-t2, indicating an increasing CBR. The slope of the CBR is negative after t3, indicating a decreasing CBR.

FIG. 5B depicts a plot 510 of an example motion-based heart rate corresponding to the calorie burn rate of FIG. 5A, consistent with step 309 of FIG. 3A. The heart rate is at HR1 from t0-t1, increases to HR2 from t1-t2, remains at HR2 from t2-t3 and begins to decrease at t3. See also FIG. 6B.

FIG. 6A depicts a relationship between activity type and calorie burn rate (CBR) consistent with step 321 of FIG. 3B. In a simplified example, different activities, e.g., Activity 1 or 2, and different intensities, e.g., 1, 2 and 3 can be associated with calorie burn rates (CBR). Calorie burn rates can be provided for repetitive activities such as certain exercises and non-repetitive activities such as sleeping and sitting. For instance, the various activities can include: cycling, calisthenics, weight lifting, rowing, aerobics, stretching, dancing, running, bowling, golf, jumping rope, skateboarding, playing tennis, swimming, gardening, cleaning, and so forth. Other activities which are not necessarily exercises can similarly be detected such as sleeping, sitting, talking and so forth. A calorie burn rate can be associated with each activity and intensity level. The calorie burn rate can be adjusted based on physiological parameters of the user as well.

FIG. 6B depicts a relationship between heart rate and calorie burn rate (CBR) consistent with step 309 of FIG. 3A. The graph depicts calorie burn rate on the horizontal axis and heart rate (e.g., motion-based heart rate) on the vertical axis. This relationship depends on physiological parameters such as gender. Typically, a male has a higher CBR than a female for a given heart rate. Other factors such as age, weight and physical condition are also relevant.

FIG. 6C depicts example accelerometer readings during an activity, consistent with step 320 of FIG. 3C. The plot depicts a relatively high amount of activity, where a repetitive pattern is detected. An accelerometer has the ability to measure acceleration in one, two or three directions, such as along the x, y and z axes of a Cartesian coordinate system. The magnitude of acceleration can be determined as well. In some cases, the acceleration is not recorded unless it exceeds threshold. A movement of a user is represented by acceleration readings, e.g., along the x, y and z axes. In one approach, each movement results in an activity count. Generally, the intensity level of activity of a user over time can be determined based on the acceleration readings. For example, amplitude, frequency and zero-crossings of the acceleration can be used to determine a level of the activity. Higher amplitudes, frequencies and zero-crossings are associated with a higher activity level.

In this example, time extends on the horizontal axis and amplitude is on the vertical axis. The detected motion is consistent with FIGS. 5A and 5B. The amplitude is from a motion sensor such as one or more accelerometers. The amplitude could represent a component (Ax, Ay, Az) along one of the x, y and z axes of an amplitude vector. Or, the amplitude could represent the magnitude of an amplitude vector, e.g., the square root of Ax̂2+Aŷ2+Aẑ3. The amplitude extends generally between A4 and A3. Acceleration readings 601 and 605 indicate small movements. In contrast, acceleration readings such as 602 and 604, with a zero crossing 603 between them, indicate larger, relatively high frequency movements. For example, the user may be running. The larger, relatively high frequency movements extend from t2-t3.

In some cases, the type of exercise that a user is performing can be detected based on characteristics of the accelerometer readings. For example, a training process may be performed in one or more users perform specified exercises and the resulting accelerometer readings are recorded. Accelerometer readings from a subsequent exercise period can be compared to the recorded accelerometer readings (signatures) to identify the exercise being performed, as well as a pace of the exercise based on the frequency of movement. For example, it may be determined that a user is running at 3 miles per hour. The type of exercise which is performed and the pace of the exercise can further be correlated with a rate of calories burned by the user based on scientific studies which have been published. The rate of calories burned can be tailored to a particular user based on physiological factors such as age, gender, height and weight. This information can all be encompassed within control logic of the processor 124 using appropriate formulas and tables.

If the type of activity cannot be detected, a general level of activity of the user can be detected (e.g., little motion, moderate motion, high motion) and a CBR associated with that level. In some cases, additional sensors such as GPS can be used to determine the current activity of a user. For example, GPS can be used to determine the speed of movement of a user.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen to best explain the principles of the technology and its practical application to thereby enable others skilled in the art to best utilize the technology in various embodiments and with various modifications as are suited to the particular use contemplated. It is intended that the scope of the technology be defined by the claims appended hereto.

What is claimed is:

1. A method for determining a heart rate of a user, comprising:
determining an energy expenditure rate of a user based on motion data from a motion sensor worn by the user;
determining a motion-based heart rate corresponding to the energy expenditure rate;
obtaining a first previous value of a heart rate of the user from a heart rate sensor worn by the user;
determining a confidence level of the first previous value of the heart rate from the heart rate sensor;
determining a spread of the motion-based heart rate, the spread is inversely proportional to the confidence level of the first previous value; and
deciding whether to use a current value from the heart rate sensor or the motion-based heart rate as a current heart rate of the user.

2. The method of claim 1, wherein:
the current value from the heart rate sensor is used as the current heart rate of the user when the current value from the heart rate sensor is within the spread; and
the motion-based heart rate is used as the current heart rate of the user when the current value from the heart rate sensor is not within the spread.

3. The method of claim 1, wherein:
the determining the confidence level of the first previous value comprises obtaining a spectrum of a time-domain signal from the heart rate sensor, and determining a magnitude of a highest peak in the spectrum, the confidence level of the first previous value is proportional to difference between the magnitude of the highest peak in the spectrum and a noise floor of the spectrum.

4. The method of claim 1, wherein:
the spread is selected from among a plurality of predetermined spreads based on the confidence level of the first previous value.

5. The method of claim 1, further comprising:
obtaining a second previous value of the heart rate of the user from the heart rate sensor, the second previous value is before the first previous value; and
determining a confidence level of the first previous value of the heart rate from the heart rate sensor, the spread is inversely proportional to the confidence level of the first previous value of the heart rate according to a first weight and to the confidence level of the second previous value of the heart rate of the user according to a second weight, and the first weight is greater than the second weight.

6. The method of claim 1, further comprising:
obtaining a plurality of previous values of the heart rate from the heart rate sensor, each value of the plurality of previous values has a respective confidence level, and the plurality of previous values comprise the first previous value; and
determining a number N of consecutive values among the plurality of previous values for which the respective confidence level exceeds a threshold, the spread is inversely proportional to the number N.

7. The method of claim 1, wherein:
the determining the energy expenditure rate comprises identifying an activity of the user.

8. The method of claim 1, wherein:
the determining the motion-based heart rate comprises determining a heart rate based on the energy expenditure rate and one or more physiological parameters of the user, using a model which is trained using heart rate values from the heart rate sensor.

9. The method of claim 1, further comprising:
determining a trend of the energy expenditure rate, the spread is relatively wider when the trend is increasing or decreasing and relatively narrower when the trend is steady.

10. The method of claim 1, wherein:
the determining the spread comprises determining a probability density function of the heart rate of the user; and
the spread comprises a dispersion metric of the probability density function.

11. The method of claim 10, wherein:
the probability density function is selected from among a plurality of predetermined probability density functions based on the confidence level of the first previous value.

12. The method of claim 10, wherein the determining the spread comprises determining a non-uniform probability density function of the heart rate of the user, the method further comprising:
obtaining a spectrum of a time-domain signal from the heart rate sensor;
determining a magnitude of a first highest peak and a magnitude of a second highest peak in the spectrum;
evaluating the non-uniform probability density function at a frequency of the first highest peak to provide a first likelihood, and obtaining a first adjusted value which is based on multiplying the magnitude of the first highest peak by the first likelihood;
evaluating the non-uniform probability density function at a frequency of the second highest peak to provide a second likelihood, and obtaining a second adjusted value which is based on multiplying the magnitude of the second highest peak by the second likelihood; and
using the frequency of the first highest peak as the current value from the heart rate sensor if the first adjusted value is greater than the second adjusted value, and using the frequency of the second highest peak as the current value from the heart rate sensor if the second adjusted value is greater than the first adjusted value.

13. A monitor, comprising:
a heart rate sensor, the heart rate sensor provides a current value and a first previous value of a heart rate of a user;
a motion sensor, the motion sensor provides motion data; and
a processor, the processor:
determines an energy expenditure rate based on the motion data;
determines a motion-based heart rate corresponding to the energy expenditure rate;
determines a confidence level of the first previous value of the heart rate;
determines a spread of the motion-based heart rate, the spread is inversely proportional to the confidence level of the first previous value; and
provides a current heart rate of the user based on at least one of the current value from the heart rate sensor or the motion-based heart rate.

14. The monitor of claim 13, wherein:
the processor selects the spread from among a plurality of predetermined spreads based on the confidence level.

15. The monitor of claim 13, wherein:
the heart rate sensor provides a second previous value before the first previous value; and
the processor determines a confidence level of the second previous value of the heart rate, and determines the spread such that the spread is inversely proportional to the confidence level of the first previous value of the heart rate according to a first weight and inversely proportional to the confidence level of the second previous value of the heart rate according to a second weight, the first weight is greater than the second weight.

16. The monitor of claim 15, wherein the processor:
obtains a second previous value of the heart rate of the user from the heart rate sensor, the second previous value is before the first previous value; and
determining a confidence level of the first previous value of the heart rate from the heart rate sensor, the spread is inversely proportional to the confidence level of the first previous value of the heart rate according to a first weight and inversely proportional to the confidence level of the second previous value of the heart rate of the user according to a second weight, and the first weight is greater than the second weight.

17. A monitor, comprising:
a heart rate sensor, the heart rate sensor provides a time-domain signal;
a motion sensor, the motion sensor provides motion data; and
a processor, the processor:
determines an energy expenditure rate of a user based on the motion data;
determines a probability density function of a heart rate using a model, the model is trained using heart rate values from the heart rate sensor, and the energy expenditure rate and is an input to the model;
obtains a spectrum of the time-domain signal from the heart rate sensor;
determines a magnitude of a first highest peak and a magnitude of a second highest peak in the spectrum;
evaluates the probability density function at a frequency of the first highest peak to provide a first likelihood, and obtains a first adjusted value which is based on multiplication of the magnitude of the first highest peak with the first likelihood;
evaluates the probability density function at a frequency of the second highest peak to provide a second likelihood, and obtains a second adjusted value which is based on multiplication of the magnitude of the second highest peak with the second likelihood; and
uses the frequency of the first highest peak as a current value of a heart rate of a user if the first adjusted value is greater than the second adjusted value, and uses the frequency of the second highest peak as the current value of the heart rate of the user if the second adjusted value is greater than the first adjusted value.

18. The monitor of claim 17, wherein:
the processor determines a trend of the energy expenditure rate; and
the probability density function is determined such that a spread of the probability density function is inversely proportional to the trend of the energy expenditure rate.

19. The monitor of claim 17, wherein:
the processor selects the probability density function from among a plurality of predetermined probability density functions based on a confidence level of at least one of the first or second highest peaks.

20. The monitor of claim 17, wherein:
the probability density function is non-uniform; and
the first likelihood differs from the second likelihood.
